(19) United States
(12) Patent Application Publication
(10) Pub. No.: US 2011/0195099 A1
(43) Pub. Date: Aug. 11, 2011

(54) PARTICULATE WAX COMPOSITES HAVING A CORE/SHELL STRUCTURE AND METHOD FOR THE PRODUCTION THEREOF AND THE USE THEREOF

(76) Inventors: Ulrich Nolte, Kleve (DE); Michael Berkei, Haltem am See (DE); Thomas Sawitcwski, Essen (DE)

(21) Appl. No.: 12/989,173

(22) PCT Filed: Mar. 26, 2009
(86) PCT No.: PCT/EP2009/002215

§ 371 Date: Apr. 11, 2011

(30) Foreign Application Priority Data
Apr. 25, 2008 (DE) 10 2008 021 005.6

(51) Int. Cl. A61K 8/02; A61Q 3/02; B32B 1/00; C08L 91/06; C09D 11/00

(52) U.S. Cl. 424/401; 424/61; 428/403; 428/405; 428/407; 106/271; 106/31.13; 977/773

(57) ABSTRACT

The invention relates to inorganic-organic composite particles having a core/shell structure, wherein the composite particles comprise an organically based core having at least one wax, and an inorganically based shell surrounding said core, and to a method for the production thereof and to the use thereof

### CROSS-REFERENCES TO RELATED APPLICATIONS

This application is a National Stage filing of International Application PCT/EP 2009/002215, filed Mar. 26, 2009, claiming priority to German Application No. DE 10 2008 021 005.6 filed Apr. 25, 2008, entitled “PARTICULATE WAX COMPOSITES HAVING A CORE/SHELL STRUCTURE AND METHOD FOR THE PRODUCTION THEREOF AND THE USE THEREOF.” The subject application claims priority to PCT/EP 2009/002215, and to German Application No. DE 10 2008 021 005.6, and incorporates all by reference herein, in their entirety.

### BACKGROUND OF THE INVENTION

The present invention relates to particulate, wax-containing composite materials (“wax composites”), and to a process for preparing them, and to the use thereof.

The present invention relates more particularly to inorganic-organic composite particles with core/shell structure, having a wax-containing core and an inorganic shell or coating surrounding said core, and to a process for preparing these composite particles, and to the use thereof.

The present invention further relates to the use of these composite particles, in particular in coating materials and coating systems, such as, in particular, paints, inks, and the like, in dispersions of all kinds, in plastics, in foams, in cosmetics, in particular nail varnishes, in adhesives, in sealants, etc.

The present invention relates, furthermore, to the use of these composite particles as fillers or ingredients, in particular in the aforementioned systems.

The present invention, finally, relates to such systems, in particular coating materials and coating systems, such as, in particular, paints, inks, and the like, plastics, foams, and cosmetics, such as, in particular, nail varnishes, which comprise these composite particles.

Additionally provided by the present invention are innovative dispersions which comprise these composite particles in a carrier medium or dispersion medium.

In order to improve the mechanical properties of coating systems and dispersion systems (including, for example, paints, inks such as printing inks, coatings) and of plastics, in particular, specifically, for increasing their wear properties, such as scratch resistance and abrasion resistance, the incorporation of additives and fillers, such as, for example, of waxes or inorganic filler particles (e.g., of what are called nanoparticles), is known in principle to she skilled person.

The inorganic filler particles known from the prior art do indeed, under certain circumstances, improve the scratch resistance of the coating systems (e.g., of paints) in which they are used; however, following application, there may be increased brittleness of the resulting coating film (e.g., of a paint film). In addition, the incorporation of these filler particles often results in unwanted clouding and deficient transparency of the coating system. In addition, relatively high filler contents are frequently necessary in order to achieve the desired effects, and this makes it more difficult to stabilize the resulting dispersion systems, and is also undesirable on cost grounds.

JP 07138484 A relates to the production of extrudates from a mixture of wax, oils or resins and a pulverulent inorganic material, such as talc or silica, for example. The incorporated additional components are said to have effects including an improved flow capacity in the extrusion operation with the wax.

JP 06166756A relates to emulsions of finely divided wax particles having particle diameters of 0.1 to 100 μm in an inert liquid, preferably a hydrofluorocarbon, such as per fluoropentane, using hydrophobic silica as emulsifier in amounts of 1 to 20 parts by weight per 100 parts by weight of wax. The hydrophobized silica, which is employed solely as an emulsifier, is obtained by reacting the surface of hydrophilic silica with a hydrophobizer, in particular halogenated alkylsilane or alkoxysilane.

JP 2004-339515 A relates to the preparation of precipitated silica having surface-modified properties, the silica prepared in this way being intended for use as a matting agent in paints. The surface modification takes place by treatment of the silica surface with a polyethylene wax, resulting in wax-coated silica particles.

KR 10-2004-0098585 A relates to precipitated silica whose surface is coated with a polyorganosiloxane polymer, and also to a process for producing it. The surface-modified silica is intended to be used as a matting agent for transparent coating materials.

Furthermore, KR 10-2005-0094496 A relates to a preparation process for a core/shell polymer latex, in order to improve the degree of coupling between wax particles and latex particles, and in that way to simplify the preparation procedure, by removing the need to prepare a wax emulsion beforehand. The preparations prepared in this way are intended to serve as toner compositions for electrophotographic image apparatus, in particular copiers.

WO 95/31508 A1 relates to wax-coated silica particles which are to be used as matting agents.

Furthermore, EP 1 182 233 E1 relates so a method of covering silicas with waxes, the intention being that the silicas described therein should find use as matting agents in paints.

EP 1 204 701 E1 relates to a cured coating on a substrate, featuring a concentration gradient of the filler particles present in the coating such that, within regions of the coating that are close to the surface, the concentration of the incorporated filler particles is greater than the concentration of these particles within the regions of the coating situated beneath. As a result of this, however, because of inhomogeneity in the coating, an improvement is achieved which is only regional, exclusively in the region of the surface.

US 2006/0228642 A1, finally, relates to a process for preparing polymer latex particles with core/shell structure, having an inner wax core and an outer latex shell, the intention being that such particles should be usable in particular for toner compositions.

### BRIEF SUMMARY OF THE INVENTION

Wax-containing composite particles for improving the mechanical properties of coating systems, in particular for increasing the wear resistance, have not yet been proposed to date in the prior art.

It is therefore an object of the present invention to provide filler particles of the aforementioned kind which are suitable in particular for use in the aforementioned systems and which at least largely avoid or else at least attenuate the disadvantages associated with the conventional particles, and also to specify a corresponding preparation process for such particles.

A further object of the present invention should be considered that of providing innovative filler particles of the type specified at the outset, which, when incorporated into the system specified at the outset, bring about an efficient performance boost and are suitable in particular for improving mechanical properties of coating systems and dispersion systems (for example, of paints, inks such as printing inks, coatings, etc.) and of plastics, in particular, especially, for increasing the wear properties thereof, in particular the scratch resistance and abrasion resistance.

in order to solve the problem outlined above, the present invention thus proposes inorganic-organic composite particles with core/shell structure.

Further provided with the present invention is a process for preparing the composite particles of the invention.

Further provided by the present invention, in turn, is the inventive use of the composite particles according to the present invention.

Further provided by the present invention, in turn, are dispersions which comprise the composite particles of the invention in a carrier medium or dispersion medium.

Additionally provided by the invention, finally, are coating materials and coating systems, in particular paints, inks, and the like, plastics, foams, cosmetics, in particular nail varnishes, adhesives, and sealants which comprise the composite particles of the invention.

### DETAILED DESCRIPTION OF THE INVENTION

It will be appreciated that, in the text below, chose remarks which relate only to one individual aspect of the present invention also apply, equally and correspondingly, to the other aspects of the present invention, without this fact requiring any explicit mention.

The present invention accordingly provides—according to a first aspect of the present invention—inorganic-organic composite particles with core/shell structure, the composite particles having an organic-based core, which comprises at least one wax, and an inorganic-based shell surrounding said core.

A particular feature of the present invention, first of all, is that inorganic-organic hybrid particles or composite particles with core/shell structure, composed of an inner wax core and an outer inorganic shell, are provided. Composite particles of this kind had not been provided to date. These particles unite the positive properties of the waxes on the one hand and of the respective inorganic shell, material on the other hand, in a single structure or in a single particle, and, on their incorporation as filler particles into coating materials and coating systems of the aforementioned kind, result in a significant improvement in the mechanical properties, in particular in an increase in the wear resistance, in particular the scratch resistance and/or the abrasion resistance.

In comparison to conventional, purely mineral or inorganic filler particles, in bulk, the amounts of the composite particles of the invention that are required, on a weight basis, in order to achieve the same performance properties are significantly lower as a result of the lower specific inherent weight. Moreover, the composite particles of the invention can be incorporated homogeneously and stably into the aforementioned systems. Their incorporation into the aforementioned systems, in particular into coating materials and coating systems, such as paints, inks, and the like, leads, as a consequence of the comparatively low refractive index, to comparatively low clouding of the systems in question.

As far as the composite particles of the invention are concerned, these composite particles generally have particle sizes of 1 to 1000 nm, in particular 5 to 800 nm, preferably 10 to 700 nm, more preferably 20 to 600 nm, very preferably 50 to 500 nm. The particle size may be determined, for example, by means of transmission electron microscopy, analytical ultracentrifugation or dynamic light scattering.

The wax-comprising core of the composite particles of the invention may have a size in the range from 1 to 400 nm, in particular 5 to 300 nm, preferably 10 to 200 nm.

It will be appreciated that, for all of the size indications and range indications specified in the context of the present invention, it may be necessary, for an individual case or for a particular application, to deviate therefrom, without departure from the scope of the present invention.

As far as the fraction of inorganic material forming the shell is concerned, this fraction may vary within wide ranges; generally speaking, the fraction of shell-forming inorganic material, based on the composite particles, is 0.5% to 80% by weight, in particular 5% to 75% by weight, preferably 10% to 70% by weight. The fraction of core-forming organic material, in particular wax, on the other hand, based on the composite particles, is generally 99.5% to 20% by weight, in particular 95% to 25% by weight, preferably 90% to 30% by weight.

As far as the shell of the composite particles of the invention is concerned, this shell may be formed of at least one optionally doped inorganic oxide (e.g., TiO₂, ZnO, Al₂O₃, SiO₂, CeO₂, Fe₂O₃, Fe₃O₄, etc.), hydroxide (e.g., Al[OH]₃ etc.), oxide hydroxide (e.g., AlOOH etc.), vanadate (e.g., BiVO₄ or YVO₄:Bi⁺, Eu³⁺), tungstate (e.g. CaWO₄), apatite, titanate, fluoride (e.g., YbF₃ or CaF₂:Eu²⁺), zeolite, sulfate (e.g., alkaline earth metal sulfates, such as barium sulfate, calcium sulfate, etc.), phosphate (e.g., alkaline earth metal phosphate, such as calcium phosphate, or lanthanum phosphate, etc.), sulfide (e.g., cadmium sulfide, zinc sulfide, etc.), carbonate (e.g., alkaline earth metal carbonate, such as magnesium carbonate, or calcium carbonate, etc.), silicate (e.g., alkaline earth metal silicate, such as calcium silicate etc.), and/or metal (e.g., silver), or else of mixtures or combinations of such compounds, or may comprise said compound(s). Advantageously the aforementioned shell material from the group of inorganic oxides, hydroxides, oxide hydroxides, sulfates, vanadates, fluorides, tungstates, phosphates, sulfides, carbonates, silicates, and/or metals is of low-solubility form in the respective medium.

In particular, the shell may be formed of at least one oxide, hydroxide, oxide hydroxide, sulfate, phosphate, sulfide, carbonate, silicate, of at least one metal or semimetal or else of metals or else of mixtures or combinations of such compounds, or may comprise said compound(s).

The shell of the composite particles of the invention may preferably be formed of at least one oxide, hydroxide and/or oxide hydroxide of aluminum, of silicon, of zinc, of titanium, of cerium and/or of iron, an alkaline earth metal sulfate, an alkaline earth metal phosphate or lanthanum phosphate, a cadmium sulfide or zinc sulfide, an alkaline earth metal carbonate, an alkaline earth metal silicate or silver, or else of mixtures or combinations of such compounds, or may comprise said compound(s).

Particular preference for forming the shell of the composite particles of the invention is given to the following compounds: TiO₂, ZnO, Al₂O₃, SiO₂, CeO₂, Fe₂O₃, Fe₃O₄, Al(OH)₃, Al(O)OH, alkaline earth metal sulfates (e.g. barium sulfate, calcium sulfate, etc.), alkaline earth metal phosphates (e.g., calcium phosphate), lanthanum phosphate, cadmium sulfide, zinc sulfide, alkaline earth metal carbonate magnesium carbonate, calcium carbonate, etc.), alkaline earth metal silicates (e.g., calcium silicate etc.) and/or silver, and also mixtures or combinations of such compounds.

It is especially preferred if the shell is formed of aluminum oxide, silicon dioxide, cerium oxide, zinc oxide and/or titanium dioxide, preferably silicon dioxide, zinc oxide and/or titanium dioxide, very preferably silicon dioxide, or comprises said compound(s).

It is even more preferred if the shell is formed of silicon dioxide (e.g., in the form of, in particular, highly disperse SiO₂ or polysilicas).

Advantageously, the shell is applied on the core at least substantially homogeneously or with at least substantially uniform layer thickness, in particular as a precipitate (i.e., as a precipitation product in the context of the preparation).

As far as the core of the composite particles of the invention is concerned, this core is formed of at least one wax or comprises such a wax. In this case the wax may in particular be selected from the group of (i) natural waxes, in particular plant, animal, and mineral waxes; (ii) chemically modified waxes; (iii) synthetic waxes; and also mixtures thereof.

As far as the concept of the wax is concerned, the term is a phenomenological designation to r a series of substances which are obtained naturally or artificially or synthetically and which in general have the following properties: waxes are kneadable at 20° C., solid to brittly hard, coarsely to finely crystalline, translucent to opaque, but not glassy, melting above 40° C. without decomposition, but being of relatively low viscosity even a short way above the melting point, and, in general and advantageously, are non-stringing, exhibit a strongly temperature-dependent consistency and solubility, and are polishable under gentle pressure. If more than one of the properties quoted above is absent, this substance, according to the DGF (Deutsche Gesellschaft für Fettwissenschaften), is not a wax (cf. DGF Standard Method M-I 1 (75)).

Waxes differ from similar synthetic or natural products (e.g., resins, plastic masses, metal soaps, etc.) primarily in that in general, approximately between 50 and 90° C., in exceptional cases even up to approximately 200° C., they undergo transition to the liquid-melt, low-viscosity state and are virtually free from ash-forming compounds.

Waxes form pastes or gels and burn generally with a sooty flame.

According to their origin, the waxes are divided into three groups, namely (i) natural waxes, including plant waxes (e.g., candelilla wax, carnauba wax, japan wax, esparto grass wax, cork wax, guaruma wax, rice germ oil wax, sugar cane wax, ouricury wax, montan wax, etc.), animal waxes (e.g., beeswax, shellac wax, spermaceti, lanolin or wool wax, uropygial grease, etc.), and mineral waxes (e.g., ceresin, ozokerite or earth wax, etc.); (ii) chemically modified waxes, including hard waxes (e.g., montan ester waxes, Sasol waxes, hydrogenated jojoba waxes, etc.); and (iii) synthetic waxes, including polyalkylene waxes, polyalkylene glycol waxes (e.g., polyethylene glycol waxes), etc.

Principal constituents of natural recent (“renewable”) waxes are esters of long-chain fatty acids (wax acids) with long-chain fatty alcohols, triterpene alcohols or steroid alcohols; these wax esters also contain free carboxyl and/or hydroxyl groups, which cause the so-called wax soaps to have emulsifying capacity. Natural fossil waxes, such as from lignite or petroleum, for example, consist primarily—like waxes from the Fischer-Tropsch synthesis or polyalkylene waxes (e.g., polyethylene waxes)—of straight-chain hydrocarbons; the former, however, depending on provenance, may also comprise branched or cycloaliphatic hydrocarbons. Frequently these “hydrocarbon” waxes are functionalized by subsequent oxidation or else, in the case of the polyolefin waxes, by comonomers with carboxyl groups.

For further details regarding the concept of waxes, reference may be made, for example, to Römpp Chemielexikon, 10th edition, volume 6, 1999, Georg Thieme Verlag Stuttgart/New York, page 4906, entry heading: “Wachse” [Waxes], and also to the literature referenced therein, especially Cosm. Toil. 101, 49 (1986), and also DGF standard methods, division M—waxes and wax products, 7th supplement 05/1999, Stuttgart: Wissenschaftliche Verlagsgesellschaft, the aforementioned literature references being hereby included by reference in their entirety in the present specification.

It is preferred in accordance with the invention if the wax comprises functional groups which are capable of interacting with the inorganic material of the shell, in particular of forming physical and/or chemical bonds therewith.

The functional groups are preferably polar groups, in particular groups which contain heteroatoms from the group of O, N and/or S, preferably O, preferably hydroxyl groups, polyether groups, in particular polyalkylene oxide groups, and/or carboxyl groups, very preferably polyether groups and/or hydroxyl groups. The functional groups of the wax forming the core of the composite particles bring about or increase the affinity of the wax material, for the inorganic shell material, and hence permit a coating of the wax core with the shell material that is, in particular, homogeneous or uniform.

As far as the organic-based core of the composite particles of the invention is concerned, this core may generally be of either uniform or single-core construction (i.e., may be composed, so to speak, of a single, homogeneous particle) or else may be composed alternatively of two or more particles or, so to speak, to have a multi-core composition.

According to one particular embodiment, provision may be made for the inorganic-based shell of the inorganic-organic composite particles of the invention to be of surface-modified design, with such surface modification taking place advantageously by means of polysiloxane groups; in other words, at or on the surface of the shell of the composite particles of the invention polysiloxane groups are applied, preferably by means of physical and/or chemical bonding, in particular chemical covalent bonding.

The corresponding surface modification by means of polysiloxane groups has the effect of an even further increase or improvement in the performance properties of the composite particles of the invention, in particular when they are incorporated as fillers into coating materials and coating systems. In particular, the surface modification, preferably with polysiloxane groups, results in reduced sedimentation propensity and gel-forming propensity of dispersions which obtain the composite Particles of the invention. In addition, embrittlement of the dried and/or cured coating system is efficiently counteracted.

A further advantage of the surface modification is that, on incorporation of the composite particles of the invention as filler particles into dispersion systems, interaction with the binder is advantageously influenced, and in this way transparency and refractive index are improved still further relative to non-surface-modified particles, and in particular, as a consequence of the reduced difference in refractive index, there is significantly less light scattering.

The surface modification, in particular by means of polysiloxane groups, is known in principle so the skilled person from the prior art. In this respect, reference may be made to the patent applications DE 10 2005 006 870 A1 or EP 1 690 902 A2 and DE 10 2007 030 285 A1 or PCT/EP 2007/006273, which originate from the applicant itself, and whose total disclosure content is hereby incorporated by reference. All aforementioned publications relate to the surface modification of metal- or semimetal-oxidic or hydroxidic surfaces by means of polysiloxanes, advantageously through formation of chemical, in particular covalent, bonds.

Further provided by the present invention—in accordance with a second aspect of the present invention—is a process for preparing the inorganic-organic composite particles of the invention with core/shell structure, as described above, where, in this process, organic-based particles which comprise or consist of at least one wax, are coated with an inorganic-based shell, and so the organic-based wax particles are surrounded by the inorganic-based shell.

As described above, the starting particles comprising wax or consisting of wax are used with a particle size in the range from 1 to 400 nm, in particular 5 to 300 nm, preferably 10 to 200 nm, and result, after having been coated with the inorganic-based material of the shell, in composite particles according to the present invention having particle sizes in the range from 1 to 1000 nm, in particular 5 to 800 nm, preferably 10 to 700 nm, more preferably 20 to 600 nm, very preferably 50 to 500 nm.

As described above, it is usual for the shell-forming inorganic material, based on the resulting composite particles, to be used in amounts of 0.5% to 80% by weight, in particular 5% to 75% by weight, preferably 10% to 70% by weight, and/or for the core-forming organic material, in particular wax, to be used, based on the resulting composite particles, in amounts of 99.5% to 20% by weight, in particular 95% to 25% by weight, preferably 90% to 30% by weight.

As far as the inorganic shell material is concerned, reference may be made, in order to avoid unnecessary repetitions, to the statements above concerning the composite particles of the invention, which apply equally in relation to the process of the invention.

As far as the wax used is concerned, reference may be made, in this regard, in order to avoid unnecessary repetitions, to the above statements concerning the composite particles of the invention, which apply correspondingly in relation to the preparation process of the invention.

In the context of the preparation process of the invention, the shell is applied to the core at least substantially homogeneously and/or with at least substantially uniform layer thickness, and this may be realized in particular with precipitation reactions. This is elucidated further in detail below.

Typically, in the context of the preparation process of the invention, the procedure is as follows: First of all, a dispersion of wax particles is provided, and subsequently the wax particles are coated with the inorganic-based shell material, the inorganic-based shell material being deposited on the wax particles in particular by means of precipitation reaction.

It is particularly advantageous in this context if a corresponding precursor of the inorganic-based shell material is used that subsequently, under reaction conditions in situ (e.g., under hydrolysis), forms the inorganic shell material and is deposited on the wax-containing cores, in particular is deposited on the wax-containing cores.

It is particularly preferred in accordance with the invention, therefore, if the inorganic-based shell material is formed in situ, in particular as part of the precipitation reaction. By way of example, the inorganic-based shell material may be formed in situ, in particular as part of the precipitation reaction, from at least one silicic ester. Silicic esters contemplated in accordance with the invention include, for example, monomeric, oligomeric or polymeric organic silicic esters, in particular of C₁-C₁₀ alcohols, more preferably alkoxysilanes having at least two functional groups, which are subsequently hydrolyzed in situ and/or reacted with the polar groups of the wax particles, and in this way are deposited, as polysilica or silicon dioxide, on the wax particles, as a shell surrounding said wax Particles. This may then be followed by a surface modification, in particular by means of polysiloxane groups, in particular in the manner described above. There may follow likewise the removal or isolation of the particles or composite particles obtained in this way.

For the reasons given above it is particularly preferred in accordance with the invention if the inorganic-based shell of the composite particles according to the invention is subjected to a surface modification, in particular by application of polysiloxane groups. For further details in this regard, reference may be made to the statements above.

In the context of the present invention it is possible in particular to use micronized waxes as core material, which are coated with inorganic shells in order to produce the composite particles of the invention. Although SiO₂ is a preferred shell or envelope material, the present invention is not however restricted to SiO₂. As described above, the surfaces of the composite particles of the invention may be surface-modified or functionalized, in particular by means of polysiloxanes, as has been described above.

Additionally provided by the present invention—according to a third aspect of the present invention—is the use of the composite particles of the invention as fillers. The composite particles of the invention can be used in particular in coating materials and coating systems, in particular paints, inks, and the like, in dispersions of all kinds, in plastics, in foams, in cosmetics, in particular nail varnishes, in adhesives, and also in sealants, in particular therein in their capacity as fillers or ingredients or additives.

The composite particles of the invention can be used in particular for contributing to improving the mechanical properties, in particular to increasing the wear resistance, preferably the scratch resistance and/or abrasion resistance, in the aforementioned systems.

Further provided by the present invention—in accordance with a fourth aspect of the present invention—are dispersions which comprise the composite particles of the invention in a carrier medium or dispersion medium.

Finally, additionally provided by the present invention—in accordance with a fifth aspect of the present invention—are coating materials and coating systems, in particular paints, inks, and the like, plastics, foams, cosmetics, in particular nail varnishes, adhesives, and sealants which comprise the composite particles of the invention.

With the composite particles of the invention, for the first time, organic-inorganic-based hybrid particles or composite particles with core/shell structure, comprising a wax-based core and an inorganic shell material, have been provided which, when incorporated into the aforementioned systems, result in a significant performance boost, in particular in a significant improvement in the mechanical properties, in particular the wear resistance, preferably the scratch resistance and/or abrasion resistance.

In comparison to mineral filler particles of the prior art which are composed of the mineral material in bulk, the composite particles of the invention have significantly lower densities or intrinsic weights. The consequence of this is that, in order to obtain comparable properties and/or effects, significantly lower weight quantities of the composite particles of the invention need to be used, in comparison to pure mineral filler particles, since the mechanical properties of the systems in question are determined by the volume fraction of the filler particles. As well as a considerable cost saving, this also results in more highly performing dispersions, which are improved in their handling as a consequence of the reduced filler content.

Furthermore, purely inorganic filler particles of the prior art have the disadvantage that they have high refractive indices as compared with a pure binder, and so their incorporation into the binders in question results in a certain clouding or reduction in gloss.

This phenomenon is not observed with the composite particles of the invention—that is, their incorporation into the binder systems in question leads to no significant clouding, since, in comparison to conventional mineral filler particles, for the reasons given above, significantly smaller quantities of the composite particles of the invention are required.

In addition, the filler particles of the invention can easily be incorporated stably, in particular with long-term stability and phase stability, into the systems in question, without any significant separation or else accumulation on the surface. As a result, the performance boost is achieved uniformly over the system as a whole.

The application possibilities for the composite particles of the invention and for the dispersions of the invention are extremely broad. The broad capacity for application in combination with the extremely high efficiency of the composite particles of the invention and of the dispersions of the invention far exceed particles and dispersions of the prior art.

The composite particles and dispersions of the invention can be employed, for example, by addition to existing systems which are processed further, for example, to give paints, adhesives, plastics, etc. Through the addition even of small quantities of the composite particles of the invention or of the dispersions of the invention, an exceptionally increased mechanical resistance is obtained.

Surprisingly, the other processing properties of the systems in question, in particular paints, plastics, etc., are not, or not significantly, influenced, and so there is no need for new optimization of the other parameters in the case of these applications.

The composite particles of the invention and dispersions thereof are therefore outstandingly suitable for use in coating materials of all kinds, plastics, adhesives, sealants, etc.

Further embodiments, modifications, and variations of the present invention are readily discernible and realizable for the skilled person from a reading of the description, without departure from the scope of the present invention.

The present invention is illustrated using the working examples which follow, and which are riot intended in any way to restrict the present invention.

### WORKING EXAMPLES

### Example 1

### Preparation of Inorganic-Organic Composite Particles with core/shell structure, Comprising Wax Core and SiO₂ Shell

The starting material, selected was a dispersion of a wax based on a polymethylalkylsilicone having polyether groups and vinyltriethoxysilane (VTEO) (hydrol.) as side chains (adduct of silicone wax±allyl-EO+C₁₈ olefin+VTEO). The aqueous emulsion was diluted with methoxypropanol in a wax emulsion/methoxypropanol ratio of 4:1, and was adjusted with water to a solids fraction of 22%.

100 g; of this mixture were heated to 40° C. and admixed with 94.0 g of TEOS (tetraethoxysilane) over a time of 8 hours with vigorous stirring. For subsequent reaction, the reaction mixture was stirred at 40° C. for two hours more.

The particle content after this reaction step was 30.7% by weight. This gave a dispersion of inorganic-organic composite particles with core/shell structure, comprising wax core and SiO₂ shell.

### Example 2

### Surface Modification by Means of Poly-Siloxanes

The functionalization of the particles from example 1 and also their transfer to an organic solvent took place in accordance with the following instructions:

60 g of the particle dispersions from example 1 were diluted with 70 g of methoxypropanol and adjusted with 1 g of ammonia solution (25% strength) to a pH >8. The mixture was then heated to 70° C., 10 mmol of propyltrimethoxysilane were added, and the mixture was stirred for two hours. Then. 80 g of methoxypropyl acetate were added, followed by removal of 63 g of solvent mixture under reduced pressure (75° C.) Subsequently, by addition of 1.22 g of a silicone (Mₙ=1000 g/mol)) with trimethoxysilyl anchor groups, with two-hour stirring, the surface of the particles prepared in example 1 was functionalized. To increase the stability of the dispersion and the compatibility in different binders, it is optionally possible to add a dispersing assistant. The particle content of the dispersion was adjusted by distillation to 17% by weight.

### Example 3

### Application Tests

The product from example 2 was added to a UV-curing clearcoat material and tested for scratch resistance. For this purpose, a particle content of 1% by weight was set in the clearcoat material, which was cured under standard conditions, the scratch resistance being determined by the implementation inter alia of a crock meter test. The same approach was carried out with pure, non-inventive SiO₂ particles in bulk.

The crock meter test was carried out as follows: Panels were coated with the corresponding filler-containing coating materials, and the coated plates were tested for scratch resistance with a crock meter instrument (model CM-5, ATLAS). For this purpose the coated plates were exposed reproducibly to a polishing cloth from the company 3M (3M polishing paper), degree of fineness, 9 μm, (10 double rubs, applied force 9 newtons). The scratch resistance was evaluated by measuring the gloss at the exposed location in comparison to the gloss of an unexposed location on the test panel. The gloss was determined using a micro-TRI-gloss measuring instrument from BYK-GARDNER, with an observation angle of 20°. The results are reproduced in the table below.

The results obtained are as follows:

| | Gloss before | Gloss after 10 crock |
|---|---|---|
| | scratching/gloss | meter cycles/gloss |
| Sample | units (GU) | units (GU) |
| Control | 87 | 40 |
| 1% by weight | 87 | 70 |
| inventive particles | | |
| from example 2 | | |
| 1.5% by weight | 87 | 80 |
| inventive particles | | |
| from example 2 | | |
| 1% by weight of | 79 | 46 |
| SiO₂ particles (not | | |
| inventive) | | |
| 2% by weight of | 75 | 55 |
| SiO₂ particles (not | | |
| inventive) | | |
| 3% by weight of | 71 | 64 |
| SiO₂ particles (not | | |
| inventive) | | |

The above results show that significant improvements can be achieved in the scratching resistance through the incorporation of the particles of the invention, the amounts required in order to bring about this effect being substantially smaller in comparison to pure SiO₂ particles in bulk in the prior art. Moreover, the incorporation of the particles of the invention in the above-specified amounts does not result in any significant clouding of the coating system, whereas, in the case of the noninventive particles, a distinct clouding even in the original coating system before scratch exposure is the result.

The tests above impressively demonstrate the enhanced performance capacity of the inventive systems and particles.

1-15. (canceled)

16. Inorganic-organic composite particles with core/shell structure, the composite particles having an organic-based core, which comprises at least one wax, and an inorganic-based shell surrounding said core,
the wax comprising functional groups which are capable of interacting with the inorganic material of the shell through formation of physical and/or chemical bonds, the functional groups being polar groups which contain heteroatoms from the group of O, N and/or S.

17. The composite particles as claimed in claim 16, wherein the composite particles have particle sizes in the range from 1 to 1000 nm.

18. The composite particles as claimed in claim 16, wherein the wax-comprising core has a size in the range from 1 to 400 nm.

19. The composite particles as claimed in claim 16, wherein the fraction of shell-forming inorganic material, based on the composite particles, is 0.5% to 80% by weight and wherein the fraction of core-forming organic material, based on the composite particles, is 99.5% to 20% by weight.

20. The composite particles as claimed in claim 16, wherein the shell is formed of at least one optionally doped dioxide, hydroxide, oxide hydroxide, sulfate, phosphate, sulfide, carbonate, silicate of at least one metal or semi-metal, or else of a metal or else of mixtures or combinations of such compounds, or comprises said compound(s).

21. The composite particles as claimed in claim 16, wherein the shell is formed of aluminum oxide, silicon dioxide, cerium oxide, zinc oxide and/or titanium dioxide, or comprises said compound(s).

22. The composite particles as claimed in claim 16, wherein the shell is formed of silicon dioxide, zinc oxide and/or titanium dioxide, or comprises said compound(s).

23. The composite particles as claimed in claim 16, wherein the shell is formed of silicon dioxide or comprises said compound.

24. The composite particles as claimed in claim 16, wherein the shell is applied on the core at least substantially homogeneously or with at least substantially uniform layer thickness.

25. The composite particles as claimed in claim 16, wherein the shell is applied on the core as a precipitate.

26. The composite particles as claimed in claim 16, wherein the core is formed of at least one wax or comprises said wax, wherein the wax is selected from the group of (i) natural waxes; (ii) chemically modified waxes; (iii) synthetic waxes; and also mixtures thereof.

27. The composite particles as claimed in claim 16, wherein the functional groups are hydroxyl groups, polyether groups, polyalkylene oxide groups, and/or carboxyl groups.

28. The composite particles as claimed in claim 16, wherein the functional groups are polyether groups and/or hydroxyl groups.

29. The composite particles as claimed in claim 16, wherein the inorganic-based shell is surface-modified.

30. The composite particles as claimed in claim 16, wherein the inorganic-based shell is surface-modified by means of polysiloxane groups.

31. A process for preparing inorganic-organic composite particles with core/shell structure as claimed in claim 1, wherein organic-based particles which comprise or consist of at least one wax are coated with an inorganic-based shell, whereby the organic-based wax particles are surrounded by the inorganic-based shell,
wherein core-forming wax used is a wax which comprises functional groups which are capable of interacting with the inorganic material of the shell by forming physical and/or chemical bonds, wherein the functional groups are polar groups which contain heteroatoms from the group of O, N and/or S.

32. The process as claimed in claim 31, wherein first a dispersion of wax particles is provided and the wax particles are subsequently coated with the inorganic-based shell material, wherein the inorganic-based shell material is deposited onto the wax particles by means of precipitation reaction, wherein the inorganic-based shell material is formed in situ as part of a precipitation reaction.

33. Dispersions comprising composite particles as claimed in claim 16 in a carrier medium or a dispersion medium.

34. A coating material comprising composite particles as claimed in claim 16.

35. The coating material of claim 34, wherein the coating material is selected from the group consisting of paints, inks, plastics, foams, cosmetics, nail varnishes, adhesives and sealants.
